(19) United States
(12) Patent Application Publication
(10) Pub. No.: US 2008/0261932 A1
(43) Pub. Date: Oct. 23, 2008

(54) METHODS AND COMPOSITIONS FOR THE PREVENTION AND/OR TREATMENT OF AN EXACERBATION OF ASTHMA

(75) Inventors: Paolo Chiesi, Parma (IT); Ivano Rondelli, Parma (IT); Daniela Acerbi, Parma (IT); Gianluigi Poli, Parma (IT)

Correspondence Address: OBLON, SPIVAK, MCCLELLAND MAIER & NEUSTADT, P.C., 1940 DUKE STREET, ALEXANDRIA, VA, 22314, US

(73) Assignee: Chiesi Farmaceutici S.p.A., Parma (IT)

(21) Appl. No.: 12/106,493

(22) Filed: Apr. 21, 2008

(30) Foreign Application Priority Data
Apr. 19, 2007 (EP) 07007930.6

(51) Int. Cl. A61K 31/56; A61P 11/06

(52) U.S. Cl. 514/171

(57) ABSTRACT

The present invention relates to the use of a composition comprising:
(a) formoterol, a pharmaceutically acceptable salt of formoterol, a solvate of formoterol, or a solvate of a pharmaceutically acceptable salt of formeterol; and
(b) beclometasone dipropionate;
   for the prevention and/or treatment of an exacerbation of asthma, intermittent asthma and/or episodes in chronic asthma during the maintenance therapy of asthma with the same composition for symptomatic relief, when needed.

### CROSS REFERENCES TO RELATED APPLICATIONS

This application claims priority to European Patent Application No. 07007930.6, filed on Apr. 19, 2006, which is incorporated herein by reference in its entirety.

### BACKGROUND OF THE INVENTION

1. Field of the Invention

The present invention relates to methods and compositions for the prevention and/or treatment of an exacerbation of asthma, intermittent asthma and/or episodes in chronic asthma. The present invention further relates to pressurized metered dose inhalers which are useful for the prevention and/or treatment of an exacerbation of asthma, intermittent asthma and/or episodes in chronic asthma.

2. Discussion of the Background

Asthma is a disease which is becoming more prevalent and is the most common disease of childhood. It can be identified by recurrent wheezing and intermittent air flow limitation. Despite many advances in its understanding, asthma remains a poorly understood and often poorly treated disease. Previously, contraction of airway smooth muscles has been regarded as the most important feature of asthma. Recently there has been a marked change in the way asthma is managed, deriving from the fact that asthma is recognized as a chronic inflammatory disease. Uncontrolled airway inflammation may lead to mucosal damage and structural changes causing irreversible narrowing of the airways and fibrosis of the lung tissue. Therapy should therefore be aimed at controlling symptoms related to airway obstruction (e.g. wheezing, dyspnea) and at the same time provide basis for treating the underlying inflammation.

The acute asthma symptoms can be relieved by first generation beta-2 adrenoceptor agonists such as salbutamol, fenoterol, and terbutalin (short-acting beta-2 agonists) or second generation ones such as formoterol and salmeterol (long-acting beta-2 agonists) which overcome the disadvantage of the short duration of action particularly for patients with nocturnal asthma.

Maintenance therapy is typically provided by anti-inflammatory glucocorticosteroids such as beclometasone dipropionate, budesonide, fluticasone propionate, mometasone furoate, and ciclesonide. Recent therapeutic strategy is aimed at both controlling the symptoms and reducing the inflammation by combinations of a long-acting beta-2 agonist and a glucocorticosteroid.

Inhalation has become the primary route of administration in the treatment of asthma. Typical delivery systems for inhalable drugs are: pressurised metered dose inhalers, dry powder inhalers, or nebulizers (ultrasonic, jet, soft-mist, etc.).

In any case, it is normal clinical practice to administer combination inhalers twice, sometimes once, daily at a dose related to the severity of asthma as a maintenance therapy and to use a short-acting beta-2 agonist, such as salbutamol or terbutaline, as required to relieve any breakthrough symptoms and acute situations, such as asthma exacerbations. These acute situations may increase in frequency and severity with loss of disease control, requiring additional administration of short-acting beta-2 agonists.

Therapy with different medications and devices may lead to poor compliance to treatment from the patients, with consequent potential under-treatment and negative impact on their quality of life. In particular the availability of a single inhaler for maintenance and rescue treatment may lead to better patient compliance to treatment and asthma control. In this regard, WO 99/64014 proposes the use of a budesonide-formoterol combination for prevention or treatment of an acute condition of asthma.

Thus, there remains a need for methods and compositions which are effective for the prevention and/or treatment of an exacerbation of asthma, intermittent asthma and/or episodes in chronic asthma.

### SUMMARY OF THE INVENTION

Accordingly, it is one object of the present invention to provide novel methods for the prevention and/or treatment of an acute condition of asthma.

It is another object of the present invention to provide novel methods for the prevention and/or treatment of an exacerbation of asthma.

It is another object of the present invention to provide novel methods for the prevention and/or treatment of intermittent asthma.

It is another object of the present invention to provide novel methods for the prevention and/or treatment of an episode of asthma.

It is another object of the present invention to provide novel methods for the prevention and/or treatment of an exacerbation of asthma, intermittent asthma and/or an episode in chronic asthma during the maintenance therapy of asthma with the same composition for symptomatic relief.

It is another object of the present invention to provide novel compositions for the prevention and/or treatment of an acute condition of asthma.

It is another object of the present invention to provide novel compositions for the prevention and/or treatment of an exacerbation of asthma.

It is another object of the present invention to provide novel compositions for the prevention and/or treatment of intermittent asthma.

It is another object of the present invention to provide novel compositions for the prevention and/or treatment of an episode of asthma.

It is another object of the present invention to provide novel compositions for the prevention and/or treatment of an exacerbation of asthma, intermittent asthma and/or an episode in chronic asthma during the maintenance therapy of asthma with the same composition for symptomatic relief.

It is another object of the present invention to provide novel dry powder inhalers, pressurized metered dose inhalers, and nebulisers which contain such a composition.

These and other objects, which will become apparent during the following detailed description, have been achieved by the inventors' discovery that administering a combination of:

(a) formoterol, a pharmaceutically acceptable salt of formoterol, a solvate of formoterol, or a solvate of a pharmaceutically acceptable salt of formeterol; and

(b) beclometasone dipropionate;

is effective for the treatment of an exacerbation of asthma, intermittent asthma and/or an episode in chronic asthma during the maintenance therapy of asthma with the same composition for symptomatic relief, when needed.

Thus, the present invention provides:

(1) a method for the prevention and/or treatment of an exacerbation of asthma, intermittent asthma and/or an episode in chronic asthma, comprising administering to a subject in need thereof an effective amount of:

(a) formoterol, a pharmaceutically acceptable salt of formoterol, a solvate of formoterol, or a solvate of a pharmaceutically acceptable salt of formeterol; and

(b) beclometasone dipropionate.

(2) A composition, comprising:

(a) formoterol, a pharmaceutically acceptable salt of formoterol, a solvate of formoterol, or a solvate of a pharmaceutically acceptable salt of formeterol; and

(b) beclometasone dipropionate.

(3) A dry powder inhaler, a pressurized metered dose inhaler, or a nebuliser, which contains:

(A) a formulation, which comprises:

(a) formoterol, a pharmaceutically acceptable salt of formoterol, a solvate of formoterol, or a solvate of a pharmaceutically acceptable salt of formeterol; and

(b) beclometasone dipropionate.

Thus, the present method relates to the administration of (a) formoterol, a pharmaceutically acceptable salt of formoterol, a solvate of formoterol, or a solvate of a pharmaceutically acceptable salt of formeterol; and (b) beclometasone dipropionate, when needed, during one or more of the following conditions:
i) an exacerbation of asthma or an acute condition of asthma is an acute asthma attack, when a patient on an irregular basis can be exposed to an agent i.e. pollen, a virus infection, cold air, exercise, perfumes or any other agent triggering an asthma attack;
ii) an intermittent asthma, i.e. in case of occasional (less than twice/week), brief exacerbations (hours to days), wherein nocturnal exacerbations are less than twice/month; or
iii) episodes, i.e. short periods of acute attacks of bronchospasms in chronic asthma.
   The present invention provides an effective combination featuring an enhanced efficacy and showing an excellent safety profile.

### DETAILED DESCRIPTION OF THE PREFERRED EMBODIMENTS

According to the present invention a combination of formoterol and beclometasone dipropionate may be administered in addition to the maintenance treatment of chronic asthma on a regular basis for the treatment of an exacerbation or acute condition of asthma, an intermittent asthma and/or episodes in chronic asthma. Moreover a combination of formoterol and beclometasone dipropionate may be administered in addition to the maintenance treatment of chronic asthma on a regular basis for the prevention of an exacerbation or acute condition of asthma, an intermittent asthma and/or episodes in chronic asthma.

In fact, in assessing the acute tolerability of high, cumulative doses of the combination formoterol and beclometasone dipropionate in comparison with formoterol alone or to a placebo it has been surprisingly found that formoterol alone caused significantly greater decrease in serum potassium level than the either the combination or the placebo. Therefore the proposed combination, even when administered in doses largely in excess than the recommended clinical dose, as in case of a rescue treatment of asthma, and in particular in case of exacerbations during the maintenance therapy of asthma, is safer than formoterol alone as shown in Example 3 below.

During the maintenance therapy of asthma with a regular administration of the present composition, i.e. twice daily, in case of exacerbations the symptoms can be counteracted by using additional doses of the same composition, thus permitting the additional glucocorticosteroid component (beclometasone dipropionate) to suppress as early as possible the enhanced airway inflammation and the additional long-acting beta-2 agonist (formoterol) to immediately reduce the bronchial constriction, minimising the risk of too frequent dosing from different inhalers.

According to the present invention the administration of the combination formoterol/beclometasone dipropionate when needed reduces the severity of exacerbations and minimizes the difficulty of a priori predicting the best dosage regimen of glucocorticosteroid (low dose or high dose) and of the short-acting or long-acting beta-2 agonist to be used in separate inhalers.

The composition of the present invention comprises a combination of formoterol and beclometasone dipropionate.

Formoterol, (±) N-[2-hydroxy-5-(1-hydroxy-2-((2-(4-methoxyphenyl)-1-methylethyl)amino)ethyl)phenyl]formamide, particularly in the form of its fumarate salt, is a bronchodilator used in the treatment of inflammatory or obstructive airways diseases. Formoterol can exist in four stereochemical forms. The present invention includes the individual stereoisomers, and in particular the (R,R)-enantiomer, as well as mixtures thereof and preferably its racemic mixture.

The formoterol may be present in the present composition as free base or as a pharmaceutically acceptable salt. Suitable pharmaceutically acceptable salts of formoterol include, for example, salts of inorganic acids such as hydrochloric, hydrobromic, sulfuric, phosphoric acids and of organic acids such as maleic, fumaric, tartaric, citric, benzoic, 4-methoxybenzoic, 2- or 4-hydroxybenzoic, 4-chlorobenzoic, p-toluenesulphonic, methanesulphonic, ascorbic, salicylic, acetic, succinic, lactic, tricarballylic, hydroxy-naphthalene-carboxylic, gluconic, and oleic acids.

The formoterol or its salts may be present in the composition in a particular crystalline form or solvated form. The preferred salt of formoterol is formoterol fumarate, particularly in the form of dihydrate.

Beclometasone dipropionate, (1-beta, 16-beta)-9-chloro-11,17,21-trihydroxy-16-methyl pregna-1,4-diene-3,20-dione 17,21-dipropionate, is a well known anti-inflammatory glucocorticosteroid, used by inhalation as an antiasthmatic compound.

The molar ratio of formoterol or pharmaceutically acceptable salt thereof to beclometasone dipropionate in the present composition, calculated as formoterol to beclometasone dipropionate, is generally from 1:1 to 1:500, preferably from 1:1 to 1:100, more preferably from 1:1 to 1:60, even more preferably from 1:3 to 1:30, most preferably from 1:12 to 1:26, and the most preferred ratio is 1:12.8 or 1:25.6.

The recommended maximum daily dose of formoterol in combination with beclometasone dipropionate for adults is 24 microgram, while the recommended maximum daily dose of beclometasone dipropionate in combination with formoterol for adults is 400 microgram. These dosages are typical for maintenance therapy.

These doses are recommended to avoid the exposure of the patient to high levels of the active compounds. However, it has been found that it is possible for the patient to administer this mixture as often as needed for a short period of time.

In particular, the daily dose of formoterol for adults, including maintenance therapy, may be as high as 168 microgram, preferably 100 microgram, and more preferably 84 microgram, and even more preferably 72 microgram.

The daily dose of beclometasone dipropionate for adults, including maintenance therapy, may be as high as 5600, preferably 2800 microgram, more preferably 2400 microgram, even more preferably 1400, and most preferably 1200 microgram.

The dose regimen will depend on the severity of the disease if mild, moderate or severe asthma and on the patient's age, sex, weight, etc.

The combination may be administered by inhalation orally or intranasally. The combination is preferably administered by a dry powder inhaler, a pressurized metered dose inhaler, or a nebuliser.

When the composition of the invention is formulated in the form of a dry powder composition to be administered by a dry powder inhaler, for example a single or a multi dose inhaler, both the active ingredients are in the form of micronized particles, preferably with a particle size of less than 10 micron.

The composition may comprise one or more suitable diluents or carriers such as lactose, dextran, mannitol or glucose. Preferably lactose is used, more preferably alpha-lactose monohydrate. Both the active ingredients of the combination of the invention and the diluent/carrier may be in a micronized form. Their mixture can optionally be subjected to agglomeration and/or spheronization.

Alternatively, a coarse diluent/carrier may be added to the composition comprising the active ingredients of the combination and optionally a diluent/carrier in the form of micronized particles, to form an ordered mixture. Said ordered mixture may optionally contain an additive to promote the release of the active ingredients. Suitable additives are substances with anti-adherent, glidant, or lubricant properties. Magnesium stearate is a particularly preferred additive.

When the composition of the invention is formulated in the form of a pressurized metered dose inhaler, the active ingredients may be both in micronized form suspended or more preferably both completely dissolved in a liquid propellant mixture. Alternatively one of the two active ingredients may be suspended and the other completely dissolved in the liquid propellant mixture.

The propellants which can be used include chlorofluorocarbons (CFCs), hydrocarbons, or hydrofluorocarbons (HFAs). Especially preferred propellants are HFA 134a (1,1,1,2-tetrafluoroethane), HFA 227 (1,1,1,2,3,3,3-heptafluoropropane) or their mixtures. The active ingredients/propellant mixtures are optionally used in combination with one or more other propellants and/or one or more additives such as cosolvents, for example ethanol, surfactants, and/or one or more lubricant, antioxidant, stabilizing and/or preserving agents such as an aqueous mineral acid and preferably 1M hydrochloric acid. When the combination of the invention is formulated in the form of a pressurized metered dose inhaler, particularly preferred are solution formulations wherein the two active ingredients of the composition are dissolved in the propellant mixture. More preferably the propellant mixture comprises and/or consists of hydrofluorocarbons, such as HFA 134a, HFA 227 or their mixtures, a cosolvent, such as ethanol and an aqueous mineral acid, such as 1M hydrochloric acid, as a stabilizing agent.

Particularly preferred are HFA solution formulations (as described in the following Examples 1, 2, and 4) which upon actuation of the pressurized metered dose inhaler, on evaporation of the propellant mixture, feature an average particle size of the two active ingredients equal or below 1.1 micrometer. These formulations are also referred herewith as extrafine formulations. Extrafine formulations assure a co-deposition in the lung areas of both the active ingredients particles. This co-deposition results in enhanced therapeutic bronchodilator effects and in a safer medicament.

In fact, due to its optimized particle distribution and increased deposition in the peripheral airways, the solution formulation combination of the present invention, allows a reduction in the glucocorticosteroid dose and a consequent reduction of its systemic exposure with respect to the marketed budesonide—formoterol dry powder inhaler formulations as described in Examples 3 and 4 of WO 99/64014. In fact according to GINA (Global Initiative for Asthma) international guidelines, Workshop Report Updated 2003, daily doses of 400 microgram of beclometasone dipropionate HFA (extrafine pressurized solution formulation) and 800 microgram of budesonide dry powder inhaler formulation are equivalent.

Therefore the availability of an extrafine HFA solution formulation of the combination of the present invention may further improve the risk/benefit ratio over the prior art formulation in the case of multiple administrations of the combination such as in the rescue treatment of asthma, and in particular in case of exacerbations during the maintenance therapy of asthma.

When the composition of the invention is formulated in the form of a nebuliser, the active ingredients may be both in micronized form suspended, dissolved, or alternatively one is dissolved and the other is suspended to give a nebulised aqueous or hydroalcoholic suspension or solution, available either as a unit dose or multi-dose formulation, with or without suitable pH or tonicity adjustment and optional addition of stabilizing and or preserving agent.

Typically, the composition is administered in total amount (including the daily maintenance regimen and the amount administered during an episode or exacerbation or to prevent an episode or exacerbation) that the dosage of the formoterol, pharmaceutically acceptable salt of formoterol, solvate of formoterol, or solvate of a pharmaceutically acceptable salt of formeterol, calculated as formoterol, is 6 to 168 microgram, alternatively 12 to 84 microgram, alternatively 24 to 72 microgram, alternatively 36 to 60 microgram, and the dosage of beclometasone dipropionate is 100 to 5600 microgram, alternatively 200 to 1400, microgram, alternatively 400 to 1200 microgram, alternatively 600 to 1000 microgram.

Other features of the invention will become apparent in the course of the following descriptions of exemplary embodiments which are given for illustration of the invention and are not intended to be limiting thereof.

### EXAMPLES

In the following examples micronization is carried out in a conventional manner such that the particle size range for each component is suitable for administration by inhalation.

### Example 1

A pressurized metered dose inhaler solution formulation contains the combination formoterol fumarate+beclometasone dipropionate contained formoterol fumarate dihydrate in an amount of 6 microgram/dose (0.010% w/w based on the total weight of the formulation), beclometasone dipropionate in an amount of 100 microgram/dose (0.172% w/w), ethanol in an amount of 12% w/w as cosolvent, and hydrochloric acid (1M) in an amount of 0.024% w/w as stabilizing agent, and HFA 134a to 100% as the propellant. The formulation is packed in aluminium cans fitted with 50 microliters valves.

### Example 2

A pressurized metered dose inhaler solution formulation contains the combination formoterol fumarate+beclometasone dipropionate contained formoterol fumarate dihydrate in an amount of 6 microgram/dose (0.008% w/w based on the total weight of the formulation), beclometasone dipropionate in an amount of 200 microgram/dose (0.271% w/w), ethanol in an amount of 12% w/w as cosolvent, and hydrochloric acid (1M) in an amount of 0.019% w/w as stabilizing agent, and HFA 134a to 100% as the propellant. The formulation is packed in aluminium cans fitted with 63 microliters valves.

### Example 3

A dry powder inhaler formulation contains the combination formoterol fumarate+beclometasone dipropionate contained micronized formoterol fumarate dihydrate in an amount of 6 microgram/dose (0.06% w/w based on the total weight of the formulation), micronized beclometasone dipropionate in an amount of 100 microgram/dose (1% w/w), 989 microgram/dose of a preblend mixture (9.89% w/w) constituted of micronized lactose and magnesium stearate (98:2 w/w), 8904 microgram/dose of coarse alpha-lactose monohydrate having a particle size comprised between 212 and 355 micron (89.04% w/w).

### Example 4

A study is performed to evaluate the tolerability of high, cumulative doses of the combination formoterol/beclometasone dipropionate or formoterol compared to a placebo when administered in asthmatic patients on regular treatment with the combination at the maximum recommended daily dose (6 microgram of formoterol/100 microgram beclometasone dipropionate, 2 puffs bid.: corresponding to a total daily dose of 24 microgram of formoterol and of 400 microgram of beclometasone dipropionate). The study is a double blind clinical comparison study of ten puffs of the pressurized metered dose inhaler solution formulation of the combination of Example 1 (formoterol 6 microgram+beclometasone dipropionate 100 microgram) or formoterol (6 microgram) or placebo during the maintenance treatment of asthma with 2 puffs bid. of the combination formoterol/beclometasone dipropionate of Example 1.

Cumulative doses (10 puffs) are administered on 3 separated days in addition to the morning dose maintenance (2 puffs). The primary endpoint is serum potassium, assessed over a 12 hours period after the cumulative doses. In addition, the effects of the treatment on the ECG (electrocardiogram), QTc (QT interval corrected of the heart's electrical cycle), blood pressure, and heart rate are evaluated at regular intervals over a 12 hours period after dosing. Plasma lactate and glucose are determined over 3 hours after dosing.

Formoterol alone causes significantly greater decrease in serum potassium level than the combination or placebo, while no significant differences in serum potassium parameters are observed between the combination and placebo. QTc, plasma lactate, and the other vital signs values for the combination are not statistically different from those with formoterol alone.

In conclusion the administration of high cumulative doses of the combination formoterol/beclometasone dipropionate in asthmatic patients on maintenance treatment with the same combination does not significantly reduce their serum potassium levels differently from formoterol alone. Therefore the combination even when administered in doses largely in excess than the recommended clinical dose, such as in case of the rescue treatment of asthma, and in particular in case of exacerbations during the maintenance therapy of asthma, is safer than formoterol in asthmatic patients.

Where a numerical limit or range is stated herein, the endpoints are included. Also, all values and subranges within a numerical limit or range are specifically included as if explicitly written out.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A method for the prevention and/or treatment of an exacerbation of asthma, intermittent asthma and/or an episode in chronic asthma, comprising administering to a patient in need thereof an effective amount of:
(a) formoterol, a pharmaceutically acceptable salt of formoterol, a solvate of formoterol, or a solvate of a pharmaceutically acceptable salt of formeterol; and
(b) beclometasone dipropionate.

2. A method according to claim 1, wherein said formoterol, a pharmaceutically acceptable salt of formoterol, a solvate of formoterol, or a solvate of a pharmaceutically acceptable salt of formeterol and said beclometasone dipropionate are administered in a molar ratio, calculated as formoterol to beclometasone dipropionate, of from 1:1 to 1:500.

3. A method according to claim 2, wherein said formoterol, a pharmaceutically acceptable salt of formoterol, a solvate of formoterol, or a solvate of a pharmaceutically acceptable salt of formeterol and said beclometasone dipropionate are administered in a molar ratio, calculated as formoterol to beclometasone dipropionate, of from 1:1 to 1:100.

4. A method according to claim 3, wherein said formoterol, a pharmaceutically acceptable salt of formoterol, a solvate of formoterol, or a solvate of a pharmaceutically acceptable salt of formeterol and said beclometasone dipropionate are administered in a molar ratio, calculated as formoterol to beclometasone dipropionate, of from 1:3 to 1:30.

5. A method according to claim 4, wherein said formoterol, a pharmaceutically acceptable salt of formoterol, a solvate of formoterol, or a solvate of a pharmaceutically acceptable salt of formeterol and said beclometasone dipropionate are administered in a molar ratio, calculated as formoterol to beclometasone dipropionate, of 1:12.8.

6. A method according to claim 4, wherein said formoterol, a pharmaceutically acceptable salt of formoterol, a solvate of formoterol, or a solvate of a pharmaceutically acceptable salt of formeterol and said beclometasone dipropionate are administered in a molar ratio, calculated as formoterol to beclometasone dipropionate, of 1:25.6.

7. A method according to claim 1, which comprises administering formoterol fumarate dihydrate.

8. A method according to claim 1, which comprises administering the R,R-enantiomer of formoterol, a pharmaceutically acceptable salt of the R,R-enantiomer of formoterol, a solvate of the R,R-enantiomer of formoterol, or a solvate of a pharmaceutically acceptable salt of the R,R-enantiomer of formoterol.

9. A method according to claim 1, wherein the daily dose of formoterol for an adult, including maintenance therapy, is as high as 168 microgram.

10. A method according to claim 1, wherein the daily dose of beclometasone dipropionate for an adult, including maintenance therapy, is as high as 5600 microgram.

11. A method according to claim 1, wherein said administering is carried out by inhalation orally or intranasally.

12. A method according to claim 11, wherein said administering is carried out with a dry powder inhaler, a pressurized metered dose inhaler, or a nebuliser.

13. A method according to claim 12, wherein said:
(a) formoterol, a pharmaceutically acceptable salt of formoterol, a solvate of formoterol, or a solvate of a pharmaceutically acceptable salt of formeterol; and
(b) beclometasone dipropionate
are formulated as a dry powder composition, which may optionally comprise one or more suitable diluents or carriers selected from the group consisting of lactose, dextran, mannitol, and glucose.

14. A method according to claim 13, wherein said:
(a) formoterol, a pharmaceutically acceptable salt of formoterol, a solvate of formoterol, or a solvate of a pharmaceutically acceptable salt of formeterol; and
(b) beclometasone dipropionate.
Are in a micronized form.

15. A method according to claim 14, wherein a coarse diluent/carrier is added to said composition to form an ordered mixture

16. A method according to claim 15, wherein said ordered mixture further comprises an additive to promote the release of the active ingredients selected from the group consisting of a substance with anti-adherent properties, a substance with glidant properties, and a substance with lubricant properties.

17. A method according to claim 12, wherein said:
(a) formoterol, a pharmaceutically acceptable salt of formoterol, a solvate of formoterol, or a solvate of a pharmaceutically acceptable salt of formeterol; and
(b) beclometasone dipropionate
are contained a pressurized metered dose inhaler and are in micronized form and suspended in a liquid propellant mixture.

18. A method according to claim 12, wherein said:
(a) formoterol, a pharmaceutically acceptable salt of formoterol, a solvate of formoterol, or a solvate of a pharmaceutically acceptable salt of formeterol; and
(b) beclometasone dipropionate
are contained in a pressurized metered dose inhaler and wherein one of said:
(a) formoterol, a pharmaceutically acceptable salt of formoterol, a solvate of formoterol, or a solvate of a pharmaceutically acceptable salt of formeterol; and
(b) beclometasone dipropionate
is suspended and the other is completely dissolved in a liquid propellant mixture.

19. A method according to claim 12, wherein said:
(a) formoterol, a pharmaceutically acceptable salt of formoterol, a solvate of formoterol, or a solvate of a pharmaceutically acceptable salt of formeterol; and
(b) beclometasone dipropionate
are contained in a pressurized metered dose inhaler and said:
(a) formoterol, a pharmaceutically acceptable salt of formoterol, a solvate of formoterol, or a solvate of a pharmaceutically acceptable salt of formeterol; and
(b) beclometasone dipropionate
are completely dissolved in a liquid propellant mixture.

20. A method according to claim 19, wherein said liquid propellant mixture comprises HFA 134a (1,1,1,2-tetrafluoroethane), HFA 227 (1,1,1,2,3,3,3-heptafluoropropane) or a mixture thereof.

21. A method according to claim 20, wherein said liquid propellant mixture further comprises at least one ingredient selected from the group consisting of a co-solvent, a surfactant, a lubricant, an antioxidant, a stabilizing agent, and a preserving agent.

22. A method according to claim 20, wherein said liquid propellant mixture comprises HFA 134a (1,1,1,2-tetrafluoroethane), ethanol, and an aqueous mineral acid.

23. A method according to claim 22, wherein said aqueous mineral acid is 1M hydrochloric acid.

24. A method according to claim 19, wherein upon actuation of said pressurized metered dose inhaler, on evaporation of the propellant mixture, an average particle size of said:
(a) formoterol, a pharmaceutically acceptable salt of formoterol, a solvate of formoterol, or a solvate of a pharmaceutically acceptable salt of formeterol; and
(b) beclometasone dipropionate
is less than or equal to 1.1 micrometer.

25. A method according to claim 12, wherein said:
(a) formoterol, a pharmaceutically acceptable salt of formoterol, a solvate of formoterol, or a solvate of a pharmaceutically acceptable salt of formeterol; and
(b) beclometasone dipropionate
are contained in a nebulizer either as a unit dose or multidose formulation.

26. A method according to claim 25, wherein said:
(a) formoterol, a pharmaceutically acceptable salt of formoterol, a solvate of formoterol, or a solvate of a pharmaceutically acceptable salt of formeterol; and
(b) beclometasone dipropionate
are in a micronized form and are suspended to give a nebulized aqueous or hydroalcoholic suspension.

27. A method according to claim 26, wherein aid:
(a) formoterol, a pharmaceutically acceptable salt of formoterol, a solvate of formoterol, or a solvate of a pharmaceutically acceptable salt of formeterol; and
(b) beclometasone dipropionate
are dissolved to give a nebulized aqueous or hydroalcoholic solution.

28. A method according to claim 25, wherein one of said:
(a) formoterol, a pharmaceutically acceptable salt of formoterol, a solvate of formoterol, or a solvate of a pharmaceutically acceptable salt of formeterol; and
(b) beclometasone dipropionate
is suspended and the other is completely dissolved to give a nebulized aqueous or hydroalcoholic suspension.
